(19) United States
(12) Patent Application Publication
(10) Pub. No.: US 2009/0220601 A1
(43) Pub. Date: Sep. 3, 2009

(54) Autologous Oral Grafts

(76) Inventor: Christopher W. Cutler, Stony Brook, NY (US)

Correspondence Address: MEDLEN & CARROLL, LLP, 101 HOWARD STREET, SUITE 350, SAN FRANCISCO, CA, 94105, US

(21) Appl. No.: 12/227,103

(22) PCT Filed: May 6, 2007
(86) PCT No.: PCT/US2007/011174

§ 371 Date: Mar. 19, 2009

Related U.S. Application Data
(60) Provisional application No. 60/798,816, filed on May 9, 2006.

(51) Int. Cl. A61K 9/14; A61K 45/00; A61P 1/02

(52) U.S. Cl. 424/484; 424/93.7

(57) ABSTRACT

The present invention provides autologous oral grafts that may be used, for example, to treat oral cavity disease characterized by inflamed or damaged oral mucosa tissue, or to treat oral caries characterized by damaged enamel, dentin, or cementum. In certain embodiments, the autologous oral grafts comprise a biocompatible matrix and cultured oral cells (e.g., keratinocytes) from the patient to be treated. In certain embodiments, the cultured oral cells comprise an expression vector comprising a nucleic acid sequence encoding a therapeutic polypeptide configured to at least partially reduce the patient's oral mucosa tissue inflammation or damage. In particular embodiments, the oral disease is characterized by infiltration of the oral mucosa with activated, maturing dendritic cells.

The present application claims priority to U.S. Provisional Application Ser. No. 60/798,816, filed May 9, 2006, which is herein incorporated by reference in its entirety.

### FIELD OF THE INVENTION

The present invention relates to autologous oral grafts that may be used, for example, to treat oral cavity disease characterized by inflamed or damaged oral mucosa tissue, including periodontitis, mucogingival defects or gingival recession or to treat oral caries characterized by damaged enamel, dentin, or cementum. In certain embodiments, the autologous oral grafts comprise a biocompatible matrix and cultured oral cells (e.g., keratinocytes) from the patient to be treated. In certain embodiments, the cultured oral cells comprise an expression vector comprising a nucleic acid sequence encoding a therapeutic polypeptide configured to at least partially reduce the patient's oral mucosa tissue inflammation or damage. In particular embodiments, the oral disease is characterized by infiltration of the oral mucosa with activated, maturing dendritic cells and other inflammatory cells.

### BACKGROUND OF THE INVENTION

There are many chronic diseases of the oral mucosa that are extremely difficult to eradicate or control, such as periodontitis, oral squamous cell carcinoma, lichen planus, oral candidasis, and verruciform xanthoma. One of the most common of these diseases is periodontitis. Inflammation or infection of the gums is called gingivitis, and, if allowed to progress, can turn into periodontitis. Periodontitis involves the inflammation of gingiva tissue and destruction of the underlying bone that anchors the teeth in place. As that happens, the gums may recede, exposing the root surfaces and increasing sensitivity to heat and cold. Gums may also recede due to other causes, including mucogingival defects. Teeth may even loosen because of bone destruction. Symptoms of periodontitis can include: bleeding gums during tooth brushing; red, swollen or tender gums; gums that have pulled away from the teeth; persistent bad breath; pus between the teeth and gums; loose or separating teeth; and a change in the way the teeth fit together when the jaw is closed. Patients with chronic generalized or localized periodontitis are routinely treated by standard initial therapy, including scaling and root planning and oral hygiene instruction, as performed by the dentist or hygienist in a dental or periodontal practice. Those that do not respond adequately after about 4-6 weeks, as evidenced by continued inflammation, bleeding on probing, loss of clinical attachment, are considered for more invasive procedures, such as gingival flap/osseous or regenerative surgical procedures. Treatment of mucogingival defects and gingival recession involves a form of gum surgery called gingival grafting, which requires the transplantation of “patches” or grafts of gingival tissue from the roof of the mouth (the “donor site”) to the site of recession (the “recipient” site). This results in a painful wound on the donor site. Alternatively, grafts are obtained from a commercial source, which harvests these grafts from the skin of an animal (xenografts, from porcine or pig). These dermal grafts are treated, sterilized, and delivered to the surgeon, who sutures them in place to the recipient site.

What is needed, therefore, are compositions, methods, and systems for treating periodontitis, and other oral diseases involving tissue inflammation and destruction, that do not rely on the invasive surgical methods or the use of foreign animal tissue currently employed and that can dampen inflammation in the long term or correct gingival recession.

### SUMMARY OF THE INVENTION

The present invention provides autologous oral grafts that may be used, for example, to treat oral cavity disease characterized by inflamed or damaged oral mucosa tissue, or recession or to treat oral caries characterized by damaged enamel, dentin, or cementum. In certain embodiments, the autologous oral grafts comprise a biocompatible matrix and cultured oral cells (e.g., keratinocytes) from the patient to be treated. In certain embodiments, the cultured oral cells comprise an expression vector comprising a nucleic acid sequence encoding a therapeutic polypeptide configured to at least partially reduce the patient's oral mucosa tissue inflammation or damage. In particular embodiments, the oral disease is characterized by infiltration of the oral mucosa with activated, maturing dendritic cells, including Langerhans cells (LC) and dermal dendritic cells (DDC).

In some embodiments, the present invention provides methods of treating oral cavity disease comprising; a) inserting an autologous oral graft into the oral cavity of a patient, wherein the oral cavity of the patient comprises oral mucosa tissue and oral hard calcified tissue (e.g., teeth), wherein a portion of the oral mucosa tissue is inflamed or damaged oral mucosa tissue as a result of an oral cavity disease or recession, or wherein a portion of the oral hard calcified tissue is damaged, and wherein the autologous oral graft comprises: i) a biocompatible matrix (e.g., membrane, collagen coated beads, etc.), and ii) cultured oral cells (e.g., keratincytes) generated from original oral cells taken from the patient, wherein the cultured oral cells comprise a first nucleic acid sequence (e.g., on an expression vector) encoding a therapeutic polypeptide; and b) attaching the autologous oral graft to the oral mucosa tissue, or to the oral hard calcified tissue, in the oral cavity of the patient such that the therapeutic polypeptide is expressed and secreted from the cultured oral cells and contacts the inflamed or damaged oral mucosa tissue or the damaged oral tissue. In particular embodiments, the cultured oral cells are grown on the biocompatible matrix. In particular embodiments, the damage to the oral mucosa tissue or the oral calcified tissue in the oral cavity of the patient is reduced or further damage is prevented (e.g., about 5% reduction in damage; about 10% reduction in damage; about 50% reduction in damage; about 99% reduction in damage; between 5-25% reduction in damage; about 25-50% reduction in damage or about 50-100% reduction in damage).

In certain embodiments, the present invention provides methods of treating oral cavity disease comprising; a) inserting an autologous oral graft into the oral cavity of a patient, wherein the oral cavity of the patient comprises oral mucosa tissue and oral hard calcified tissue, wherein a portion of the oral mucosa tissue is inflamed or damaged oral mucosa tissue as a result of an oral cavity disease or wherein a portion of the oral calcified tissue is damaged, and wherein the autologous oral graft comprises: i) a biocompatible matrix, and ii) cultured oral keratinocytes generated from oral keratinocytes taken from the patient; and b) attaching the autologous oral graft to the oral mucosa tissue, or the oral hard calcified tissue, in the oral cavity of the patient such that the damage to the oral mucosa tissue or the oral calcified tissue in the oral cavity of the patient is reduced or further damage is prevented (e.g., about 5% reduction in damage; about 10% reduction in damage; about 50% reduction in damage; about 99% reduction in damage; between 5-25% reduction in damage; about 25-50% reduction in damage or about 50-100% reduction in damage).

In additional embodiments, the present invention provides methods of treating dental caries comprising; a) inserting an autologous oral graft into the oral cavity of a patient, wherein the oral cavity of the patient comprises oral mucosa tissue and a plurality of teeth, wherein at least one tooth has dental caries, and wherein the autologous oral graft comprises: i) a biocompatible matrix, and ii) cultured oral keratinocytes generated from original oral keratinocytes taken from said patient (e.g., from the oral mucosa tissue), wherein the cultured oral keratinocytes optionally comprise an expression vector comprising a first nucleic acid sequence encoding a therapeutic polypeptide for dental caries; and b) attaching the autologous oral graft to the oral mucosa tissue, or to a tooth, in the oral cavity of said patient such that the therapeutic polypeptide is expressed and secreted (or endogenous therapeutic factors are expressed and secreted) from the cultured oral keratinocytes and contacts the at least one tooth having dental caries. In further embodiments the dental caries is characterized by damaged hard calcified tissue (e.g., damaged tooth enamel, dentin or cementum).

In certain embodiments, the amount of inflamed or damaged oral mucosa tissue in the oral cavity of the patient is reduced as a result of being contacted with the therapeutic polypeptide (e.g. there is a 10%, or 20%, or 40%, or 50%, or 75% reduction in the amount of inflammation or damaged tissue in the oral cavity of the patient). In some embodiments, the biocompatible matrix comprises a biocompatible membrane. In particular embodiments, the oral mucosa tissue has been inflamed by infiltration of the oral mucosa with activated, maturing dendritic cells, including Langerhans cells (LC) and/or dermal dendritic cells (DDC) and T cells.

In certain embodiments, the cultured oral cells (e.g., oral keratinocytes) further comprise a second nucleic acid sequence encoding a reporter protein, wherein the reporter protein allows monitoring of expression of the therapeutic polypeptide. In particular embodiments, the reporter protein allows non-invasive monitoring of expression of the therapeutic polypeptide (e.g., expression of the therapeutic polypeptide can be monitored without removing the autologous oral graft from its position on the oral mucosal tissue, or oral hard calcified tissue, of the patient). In additional embodiments, the methods further comprise detecting expression of the reporter protein without removing the autologous oral graft from the oral mucosa tissue thereby determining if the therapeutic polypeptide is expressed. In particular embodiments, the reporter protein is selected from green fluorescent protein (GFP), firefly luciferase (FL), and beta-glucuronidase. In further embodiments, expression of the reporter protein is monitored, without removing the autologous oral graft from the oral tissue, with a light detection cooled charge coupled device (CCCD) or with a UV light, or with a fluorescence detector. In certain embodiments, the second nucleic acid sequence encoding the reporter protein is located on the same expression vector as the first nucleic acid sequence encoding the therapeutic polypeptide.

In particular embodiments, the autologous oral graft further comprises a second type of oral cells (e.g., cultured fibroblasts), wherein the second type of cells are generated from original cells taken from the patient. In certain embodiments, the cultured second type of cells (e.g., oral fibroblasts) form a first layer on top of the biocompatible matrix and the transfected first type of cells (e.g., cultured and transfected oral keratinoctyes) form a second layer on top of the first layer.

In some embodiments, the oral cavity disease is selected from the group consisting of: chronic periodontitis, oral squamus cell carcinoma, lichen planus, oral yeast infections, oral cancer, oral candidasis verruciform xanthoma, gingival recession and dental caries. In particular embodiments, the oral cavity disease is periodontitis.

In other embodiments, the therapeutic polypeptide comprises an anti-inflammatory cytokine (e.g., those cytokines that are able to inhibit the synthesis of IL-1, tumor necrosis factor (TNF), and other major proinflammatory cytokines or cytokine that can shift the T cell effector response towards Th1 (e.g. IFNγ, IL-12) or Th2 (e.g. IL-10, IL-4, IL-13)). In particular embodiments, the therapeutic polypeptide comprises an inhibitor of soft tissue proteolysis or inhibitor of matrix metalloproteinases (e.g., TIMP-1, TIMP-2, TIMP-3, or TIMP-4). In other embodiments, the therapeutic polypeptide comprises an inhibitor of bone resorption (e.g. OPN). In some embodiments, the therapeutic polypeptide comprises a stimulator of bone or other hard or soft tissue growth or regeneration (e.g. parathyroid hormone (PTH), bone morphogenetic protein (BMP), platelet-derived growth factor (PDGF-AB, PDGF-BB), transforming growth factor-beta1 (TGFβ-1), insulin-like growth factor-I (IGF-1), fibroblast growth factor-basic (FGF-b), epidermal growth factor (EGF), vascular endothelial growth factor (VEGF). In other embodiments that therapeutic polypeptide comprises a negative regulator of intracellular signaling (e.g. SHIP, SOCS, and IRAK-M). In further embodiments, the therapeutic polypeptide comprises an inhibitor of DC/T cell activation (e.g., CTLA-4). In some embodiments, the first nucleic acid sequence encodes a therapeutic polypeptide selected from the group consisting of: IL-10, TGF-β, IL-1RA, vasoactive intestinal peptide (VIP), CTLA-4, SHIP, SOCS-1, and IRAK-M. In some embodiments, the therapeutic polypeptide comprises an inhibitor of bacterial enzymes such glucosyltransferases and serine/threonine protein kinases involved in biofilm formation and in dental caries (e.g. CovR, a negative regulator of glucosyltransferase in S. mutans) or inhibitors of bacterial colonization (e.g. S. mutans-specific secretory IgA). In certain embodiments, the inflamed or damaged oral mucosa tissue is selected from the group consisting of: palate tissue, gingiva tissue, buccal mucosa tissue, tongue tissue, and floor of the mouth tissue. In other embodiments, the inflamed or damaged oral mucosa tissue comprises gingiva tissue. In further embodiments, the inflamed or damaged oral mucosa tissue is inflamed tissue.

In some embodiments, at least a portion of the cultured oral cells (e.g., oral keratinoctyes) migrate from the autologous oral graft to the oral mucosa of the patient. In other embodiments, at least a portion of the cultured oral cells migrate from the autologous oral graft to the oral mucosa of the patient, and the presence of the cultured oral cells in the oral mucosa is determined by detecting expression of a reporter protein in the oral mucosa. In certain embodiments, the cultured oral cells integrate into the oral mucosa and continue to produce the therapeutic polypeptide.

In particular embodiments, the biocompatible matrix is bioresorbable. In further embodiments, the biocompatible matrix comprises polylactic acid. In other embodiments, the biocompatible matrix comprises collagen or ALLODERM regenerative tissue matrix (LifeCell Corp., Branchburg N.J.). In additional embodiments, the biocompatible matrix comprises pores. In further embodiments, the pores have an average size of about 2-25 μm (e.g., 2 . . . 8 . . . 12 . . . 17 . . . 21 . . . or 25 μm). In other embodiments, the biocompatible matrix has a length between about 10 mm and 50 mm (e.g., 10 . . . 20 . . . 30 . . . 40 . . . or 50 mm). In further embodiments, the biocompatible matrix has a width between about 3 mm and 20 mm (e.g., 3 . . . 7 . . . 11 . . . 15 . . . or 20 mm). In other embodiments, the biocompatible matrix has a thickness between about 0.03 mm and 0.45 mm (e.g., 0.03 . . . 0.07 . . . 0.10 . . . 0.20 . . . 0.30 . . . and 0.45 mm). In particular embodiments, the biocompatible matrix comprises a plurality of preformed suture holes (e.g., 2, 3, 4, 5, 6, 7, 8, 9, 10, or more pre-formed suture holes). In particular embodiments, the patient is a human.

In certain embodiments, the present invention provides methods of making an autologous oral graft comprising; a) culturing original oral cells (e.g., keratinoctyes) taken from the oral cavity of a patient to generate cultured oral cells, wherein the oral cavity of the patient comprises oral mucosa tissue and oral hard calcified tissue, wherein a portion of the oral mucosa tissue is inflamed or damaged oral mucosa tissue as a result of an oral cavity disease, or a portion of the oral hard calcified tissue is damaged (e.g., the tooth enamel or dentin or cementum is damaged as a result of oral infection with cariogenic bacteria) and wherein the original oral cells are taken from a portion of the oral mucosa tissue that is not inflamed or damaged, b) transfecting the cultured oral cells with an expression vector comprising a first nucleic acid sequence encoding a therapeutic polypeptide, and c) combining the cultured oral cells with a biocompatible matrix to generate an autologous oral graft, wherein the autologous oral graft is configured to be attached to the oral mucosa tissue or oral hard calcified tissue of the patient such that the therapeutic polypeptide is expressed and secreted from the cultured oral cells and contacts the inflamed or damaged oral mucosa tissue or the damaged oral hard calcified tissue (e.g., tooth enamel or dentin or cementum).

In some embodiments, the present invention provides methods of making an autologous oral graft comprising; a) culturing oral keratinocytes taken from the oral cavity of a patient to generate cultured oral keratinocytes, wherein the oral cavity of the patient comprises oral mucosa tissue and oral hard calcified tissue, wherein a portion of the oral mucosa tissue is inflamed or damaged oral mucosa tissue as a result of an oral cavity disease, or wherein a portion of the oral hard calcified tissue is damaged, and wherein the oral keratinocytes are taken from a portion of the oral mucosa tissue that is not inflamed or damaged, and b) combining the cultured oral keratinocytes with a biocompatible matrix to generate an autologous oral graft, wherein the autologous oral graft is configured to be attached to the oral mucosa tissue, or the oral hard calcified tissue, of the patient such that the damage to the oral mucosa tissue or the oral calcified tissue in the oral cavity of the patient is reduced (e.g., about 5% reduction in damage; about 10% reduction in damage; about 50% reduction in damage; about 99% reduction in damage; between 5-25% reduction in damage; about 25-50% reduction in damage or about 50-100% reduction in damage).

In certain embodiments, the present invention provides methods of making an autologous oral graft comprising; a) culturing original oral cells (e.g., keratinocytes) taken from the oral cavity of a patient to generate cultured oral cells, wherein the oral cavity of the patient comprises oral mucosa tissue and oral hard calcified tissue, wherein a portion of the oral mucosa tissue is inflamed or damaged oral mucosa tissue as a result of an oral cavity disease, or a portion of the oral hard calcified tissue is damaged, and wherein the original oral cells are taken from a portion of the oral mucosa tissue that is not inflamed or damaged, b) combining the cultured oral cells with a biocompatible matrix to generate an autologous oral graft, wherein the autologous oral graft is configured to be attached to the oral mucosa tissue, or oral hard calcified tissue, of the patient, and, in certain embodiments, step c) transfecting the cultured oral cells with a first nucleic acid sequence (e.g., as part of an expression vector) encoding a therapeutic polypeptide.

In certain embodiments, the present invention provides methods of storing patient biological samples or oral tissues, in an oral tissue bank, so that they can later be thawed and used, in the event that further grafting procedures are needed or in the event that the procedures need to be repeated.

In some embodiments, the methods further comprise the step of culturing a second type of original oral cells taken from the oral cavity of the patient to generate a second type of cultured oral cells (e.g., oral fibroblasts). In further embodiments, the second type of cultured oral cells are combined with the cultured (and, in certain embodiments, transfected) oral cells and the biocompatible matrix to generate the autologous oral graft. In other embodiments, the second type of cultured oral cells form a first layer on top of the biocompatible matrix and the cultured oral cells form a second layer on top of the first layer. In particular embodiments, the present invention provides autologous oral grafts made by the methods described herein.

In particular embodiments, the present invention provides methods for making an autologous oral graft comprising; a) removing a biological sample from the oral cavity of a patient, wherein the biological sample comprises original oral cells (e.g., keratinocytes), wherein the oral cavity of the patient comprises oral mucosa tissue and oral hard calcified tissue, wherein a portion of the oral mucosa tissue is inflamed or damaged oral mucosa tissue as a result of an oral cavity disease or a portion of the oral hard calcified tissue is damaged, and wherein the biological sample is taken from a portion of the oral mucosa tissue that is not inflamed or damaged, b) culturing the original oral cells from the tissue sample to generate cultured oral cells, c) transfecting the cultured oral cells with a first heterologous nucleic acid sequence (e.g., as part of an expression vector) encoding a therapeutic polypeptide, and d) combining the cultured oral cells with a biocompatible matrix to generate an autologous oral graft, wherein the autologous oral graft is configured to be attached to the oral mucosa tissue of the patient or the oral hard calcified tissue such that the therapeutic polypeptide is expressed and secreted from the cultured oral cells and contacts the inflamed or damaged oral mucosa tissue or the damaged oral hard calcified tissue. In certain embodiments, the removing comprises cutting a portion of the oral mucosa tissue with a scalpel or other device for removing a sample of tissue. In other embodiments, the biological sample is rinsed with a buffer comprising antibiotics. In further embodiments, the biological sample, before step b), is frozen in liquid nitrogen. In some embodiments, the present invention provides autologous oral grafts made by the above method.

In certain embodiments, the present invention provides methods of making an autologous oral graft comprising; a) receiving a biological sample from a patient or a patient's health care provider, wherein the biological sample is from the oral cavity of the patient, wherein the biological sample comprises original oral cells (e.g., keratinocytes), wherein the oral cavity of the patient comprises oral mucosa tissue and oral hard calcified tissue, wherein a portion of the oral mucosa tissue is inflamed or damaged oral mucosa tissue as a result of an oral cavity disease or recession, or a portion of the oral hard calcified tissue is damaged (e.g., tooth enamel or dentin or cementum is damaged as a result of oral infection with cariogenic bacteria) and wherein the biological sample is taken from a portion of the oral mucosa tissue that is not inflamed or damaged, b) culturing the original oral cells from the biological sample to generate cultured oral cells, and optionally the step of c) transfecting the cultured oral cells with a first nucleic acid sequence (e.g. as part of an expression vector) encoding a therapeutic polypeptide, d) combining the cultured oral cells with a biocompatible matrix to generate an autologous oral graft, wherein the autologous oral graft is configured to be attached to the oral mucosa tissue or oral hard calcified tissue of the patient, and e) shipping the autologous oral graft such that it is received by the patient or the patient's health care provider. In some embodiments, the autologous oral graft is configured to be attached to the oral mucosa tissue or oral hard calcified tissue of the patient such that the therapeutic polypeptide is expressed and secreted from the cultured oral keratinocytes and contacts the inflamed or damaged oral mucosa tissue or damaged oral hard calcified tissue (e.g., damaged tooth enamel or dentin or cementum).

In particular embodiments, the shipping comprises mailing the autologous oral graft to the patient, the patient's health care provider, or to an intermediary that mails (or otherwise delivers) the autologous oral graft to the patient's health care provider. In other embodiments, the receiving, the shipping, or both the receiving and the shipping are performed by a graft manufacturing facility. In additional embodiments, the patient's health care provider is selected from the group consisting of: an HMO, a hospital, a doctor, a dental office, a dentist, and a dental surgeon. In other embodiments, the receiving and/or shipping is via mail, courier, hand delivery, or combinations thereof.

In particular embodiments, the present invention provides the autologous oral grafts made by the above methods described herein.

In some embodiments, the present invention provides methods of making an autologous oral graft comprising; a) attaching a biocompatible matrix at or near a distal end of a tube (e.g., that is square or rectangular or any other shape) that is initially open at both ends in order to generate a bioreactor cup having the biocompatible matrix as the floor of the bioreactor cup, b) adding a second type of oral cells (e.g., fibroblasts) on top of the biocompatible matrix inside the bioreactor cup such that a layer of second oral cells is generated, wherein the second oral cells are original or cultured oral cells from the oral cavity of a patient, wherein the oral cavity of the patient comprises oral mucosa tissue and oral hard calcified tissue, wherein a portion of the oral mucosa tissue is inflamed or damaged oral mucosa tissue as a result of an oral cavity disease or a portion of the oral hard calcified tissue is damaged, c) adding a first type of oral cells (e.g., keratinocytes) on top of the layer of second type of oral cells inside the bioreactor cup, wherein the first type of oral cells are cultured oral cells from the oral cavity of the patient, wherein the first type of oral cells comprise an expression vector comprising a first nucleic acid sequence encoding a therapeutic polypeptide, and d) incubating the cup such that an autologous oral graft is generated inside the cup, wherein the autologous oral graft is configured to be attached to the oral mucosa tissue or oral hard calcified tissue of the patient such that the therapeutic polypeptide is expressed and secreted from the first type of cultured oral cells and contacts the inflamed or damaged oral mucosa or damaged oral hard calcified tissue. In certain embodiments, the tube comprises polypropylene. In other embodiments, the tube is a square-or rectangular shaped tube. In further embodiments, the methods further comprise excising the autologous oral graft from the cup by cutting the cup.

In further embodiments, the present invention provides the cup produced according the methods described above. In other embodiments, the present invention provides the autologous oral graft produced by the above methods.

In some embodiments, the present invention provides autologous oral grafts configured for use in the oral cavity of a patient with oral disease characterized by inflammation or destruction of oral mucosa tissue or damaged oral hard calcified tissue comprising; a) a biocompatible matrix comprising pores, wherein the biocompatible matrix: i) is between about 0.05 mm and 0.30 mm thick, ii) is between about 15 mm and about 35 mm long; and iii) is between about 5 mm and about 15 mm wide, and b) cultured oral cells (e.g., keratinocytes) from the oral cavity of the patient, wherein the cultured oral cells comprise an expression vector comprising a first nucleic acid sequence encoding a therapeutic polypeptide, wherein the therapeutic polypeptide is configured to reduce inflammation or tissue destruction of the oral mucosa tissue or reduce or prevent further damage to the oral hard calcified tissue.

In certain embodiments, the autologous oral graft farther comprises a layer of second oral cells (e.g., fibroblasts) on top of the biocompatible matrix, wherein the second oral cells are original or cultured oral cells from the oral cavity of the patient. In some embodiments, the cultured oral cells form a layer of cultured oral cells on top of the layer of second type of oral cells.

In particular embodiments, the pores have an average size between about 3 and about 15 μm. In further embodiments, the biocompatible matrix comprises a plurality of pre-formed suture holes. In some embodiments, the biocompatible matrix is bioresorbable. In additional embodiments, the biocompatible matrix comprises polylactic acid.

In some embodiments, the present invention provides systems comprising; a) an autologous oral graft comprising: i) a biocompatible matrix, and ii) cultured oral cells (e.g., keratincytes) generated from original oral cells taken from a patient, wherein the cultured oral cells comprise a first nucleic acid sequence (e.g., as part of an expression vector) encoding a therapeutic polypeptide; and b) a first sealed container, wherein the autologous oral graft is located inside the sealed container.

In particular embodiments, the present invention provides systems comprising; a) an autologous oral graft comprising: i) a biocompatible matrix, and ii) cultured oral cells (e.g., keratincytes) generated from original oral cells taken from a patient, and b) a first sealed container, wherein the autologous oral graft is located inside the sealed container.

In further embodiments, the system further comprises c) a temperature indicator, wherein the temperature indicator visually indicates if the first sealed container has been exposed to a threshold temperature at any point. In further embodiments, the temperature indicator is a WARMMARK temperature indicator from IntroTech (Loenen, Netherlands). In particular embodiments, the systems further comprise a second sealed container, wherein the first sealed container is located inside of the second sealed container. In other embodiments, the systems further comprise second sealed container, wherein the first sealed container is located inside the second sealed container and wherein the temperature indicator is affixed to the second sealed container. In certain embodiments, the first sealed container comprises a biocompatible material.

In some embodiments, the present invention provides systems comprising; a) a dental office, wherein the dental office is caring for a patient, wherein the patient has an oral cavity comprising oral mucosa tissue and hard oral calcified tissue, wherein a portion of the oral mucosa tissue is inflamed or damaged oral mucosa tissue or a portion of the hard oral calcified tissue is damaged as a result of an oral cavity disease, and b) an autologous oral graft comprising; i) a biocompatible matrix, and ii) cultured oral cells generated from original oral cells taken from the patient. In certain embodiments, the cultured oral cells comprise an expression vector comprising a first nucleic acid sequence encoding a therapeutic polypeptide.

In certain embodiments, the autologous oral graft is located inside the dental office (e.g. after being shipped to the dental office from a graft manufacturing facility). In other embodiments, the autologous oral graft further comprises a second type of cultured oral cells (e.g., fibroblasts), wherein the second type of oral cells are generated from original cells taken from the patient.

In some embodiments, the present invention provides methods of treating dental caries comprising; a) inserting an autologous oral graft into the oral cavity of a patient, wherein the oral cavity of the patient comprises oral mucosa tissue and a plurality of teeth, wherein at least one tooth has dental caries (tooth decay) as a result of acids secreted by Streptococcus mutans and/or Lactobacilli, and wherein the autologous oral graft comprises: i) a biocompatible matrix, and ii) cultured oral cells (e.g., keratinocytes) generated from original oral cells taken from the patient, wherein the cultured oral cells comprise a first nucleic acid sequence (e.g., as part of an expression vector) encoding a therapeutic polypeptide; and b) attaching the autologous oral graft to the oral mucosa tissue, or to at least one tooth, in the oral cavity of the patient such that the therapeutic polypeptide is expressed and secreted from the cultured oral cells and contacts the at least one tooth having dental caries. In certain embodiments, the therapeutic polypeptide is configured to disrupt or stop the ability of S. mutans and/or Lactobacilli from producing acid. In other embodiments, the therapeutic polypeptide comprises at least a portion of salivary secreted IgA (e.g. a Fab fragment of salivary secreted IgA).

### DESCRIPTION OF THE FIGURES

FIG. 1 shows an exemplary autologous oral graft system, including one embodiment for preparing the autologous oral graft (FIGS. 1A-1C) and one embodiment for treating inflamed oral mucosa involved in chronic periodontitis with the autologous oral graft (FIGS. 1D-1G).

FIG. 2A shows a closed vial containing an autologous oral graft, along with a scalpel. FIG. 2B shows the autologous oral graft after being cut away from the closed vial using the scalpel. FIG. 2C shows the autologous oral graft attached to the oral mucosa of a patient.

### DEFINITIONS

To facilitate an understanding of the invention, a number of terms are defined below.

As used herein, the terms “subject” and “patient” refer to any animal, such as a mammal like livestock, pets, and preferably a human. Specific examples of “subjects” and “patients” include, but are not limited to, individuals with damaged oral hard calcified tissue (e.g. dental caries) or inflamed or damaged oral mucosa tissue caused by gingival recession or by an oral cavity disease such as: chronic periodontitis, oral squamus, lichen planus, oral yeast infections, and oral candidasis verruciform xanthoma.

As used herein, the term “oral mucosa” refers to the mucous matrix that covers all structures inside the oral cavity except the teeth. The oral mucosa generally varies in color from pink to brownish purple depending on an individual's skin color. The structure of the oral mucosa varies depending on its location in the oral cavity and the function of that area. For example, the mucosa lining the cheeks is not designed to withstand the heavy force of mastication while the masticatory mucosa covering the jaws is structured to withstand the forces of mastication. A specialized mucosa that includes taste buds covers the tongue. Example of oral mucosa tissue include, but are not limited to, palate tissue, gingiva tissue, buccal mucosa tissue, tongue tissue, and floor of the mouth tissue.

As used herein, the phrase “oral hard calcified tissue” refers to the hard tissue that makes up teeth, including, but not limited to: enamel, dentin, and cementum.

As used herein, the term “biocompatible matrix” refers to any biocompatible three dimensional network of materials, including, but not limited to, extracellular matrices, synthetic or biological polysaccharide matrices, collagen matrices, matrigel, polylactic acid, ALLODERM, polymer networks, soft microfabricated structures (e.g., from PDMS), gels of lyotropic liquid crystals, and matrices prepared from bacterial cell secretions. The materials of the matrices may be chemically crosslinked or physically crosslinked.

As used herein the term “polypeptide” is used in its broadest sense to refer to all molecules or molecular assemblies containing two or more amino acids. Such molecules include, but are not limited to, proteins, peptides, enzymes, antibodies, receptors, lipoproteins, and glycoproteins.

The term “transfection” as used herein refers to the introduction of foreign nucleic acid into eukaryotic cells. Transfection may be accomplished by a variety of means known to the art including, but not limited to, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The phrase “receiving a biological sample from a patient or a patient's health care provider” refers to an entity taking possession of a patient's biological sample that has been provided by the patient or patient's health care provider. For example, an entity, such as a company, may receive a patient's biological sample by mail, courier, or may draw the sample from the patient themselves. These biological samples or tissues may be cultured and used immediately or stored at an “oral tissue graft bank” for later thawing if the need arises to repeat the procedure, without the need for obtaining another clinical sample from the patient.

As used herein, the term “bioreactor cup” refers to any container capable of holding liquid (e.g., growth media) that has a biocompatible matrix forming at least part of the floor of the container. As used herein, the phrase “sealed cup” refers to a bioreactor cup that has been closed such that any liquid inside is substantially prevented from exiting regardless of the position of the sealed cup.

As used herein, the phrase “sealed container” refers to any enclosure that is capable of housing an autologous oral graft of the present invention such that the autologous oral graft cannot escape without tearing the sealed container. Examples of sealed containers include, but are not limited to, cardboard boxes, metal foil lined pouches, and plastic vials.

As used herein, the phrase “pre-formed suture holes” refers to holes that are located in the biocompatible matrix that can be used to suture the final autologous oral graft in place in the oral cavity of a patient. The holes can be formed, for example, at the time the biocompatible matrix is generated (e.g. as part of a mold) or they can be cut into the biocompatible matrix with a cutting tool.

The term “expression vector” as used herein refers to a recombinant nucleic acid molecule containing a desired coding sequence and appropriate nucleic acid sequences necessary for expression of the operably linked coding sequence (e.g. insert sequence that codes for a product) in a particular host organism.

As used herein, the phrase “anti-inflammatory cytokine” refers to those cytokines with the ability to inhibit the synthesis or activity of proinflammatory cytokines, such as IL-1 and TNF. Examples of anti-inflammatory cytokines include, but are not limited to, interleukin (IL)-1 receptor antagonist, IL-4, IL-6, IL-10, IL-11, IL-13, and TGF-β (see, also, Opal et al., Chest. 2000; 117:1162-1172, herein incorporated by reference).

The term “reporter gene” indicates a gene sequence that encodes a “reporter polypeptide.” A “reporter polypeptide” is detectable in any detection system, including, but not limited to enzyme (e.g., ELISA, as well as enzyme-based histochemical assays), fluorescent, radioactive, and luminescent systems. In certain embodiments, the present invention contemplates the E. coli beta-galactosidase gene (available from Pharmacia Biotech, Pistacataway, N.J.), green fluorescent protein (GFP) (commercially available from Clontech, Palo Alto, Calif.), the human placental alkaline phosphatase gene, the chloramphenicol acetyltransferase (CAT) gene, or other reporter genes are known to the art.

### GENERAL DESCRIPTION OF THE INVENTION

The present invention provides autologous oral grafts that may be used, for example, to treat oral cavity disease characterized by inflamed or damaged oral mucosa tissue or recession or damaged oral hard tissue (e.g. dental caries). In certain embodiments, the autologous oral grafts comprise a biocompatible matrix and cultured oral cells (e.g., keratinocytes) from the patient to be treated. Preferably, the cultured oral cells comprise an expression vector comprising a nucleic acid sequence encoding a therapeutic polypeptide configured to at least partially reduce the patient's oral mucosa tissue inflammation or damage. In particular embodiments, the oral disease is characterized by infiltration of the oral mucosa with activated, maturing dendritic cells, including Langerhans cells (LC) and dermal dendritic cells (DDC) and T cells.

Previous work has established that chronic periodontitis is an inflammatory disease characterized by infiltration of the oral mucosa (e.g., gums) with activated, maturing dendritic cells, including Langerhans cells (LC) and dermal dendritic cells (DDC) (See, Cutler et al 1999, Adult Periodontitis. J. Perio Res. 34:406-412; Jotwani et al 2001, J. Immunol. 167: 4693-4700; Jotwani et al 2003, J. Dental Res. 82:736-741; Cutler and Jotwani, 2004, Periodontitis Periodontology 2000, 35:135-157; Cirrincione et al. 2002, J Periodontol, 2002 January; 73(1):45-52; Mahanonda et al., J Periodontal Res. 2002 June; 37(3):177-83; Aroonrerk et al. J Periodontal Res. 2003 June; 38(3):262-8; and Gemmell et al., Oral Microbiol Immunol. 2002 December; 17(6):388-93, all of which are herein incorporated by reference in their entireties as if fully set forth herein). These infiltrating dendritic cells are potent antigen presenting cells that form immune conjugates locally with CD4+ T cells, eliciting a cell mediated inflammatory response that results, ultimately, in tissue and bone destruction. As such, in preferred embodiments, the autologous oral grafts of the present invention are used to dampen this local inflammatory response and preserve soft and hard tissue that support the teeth. In particular embodiments, the autologous oral grafts are used to blunt activation and maturation of antigen presenting cells and inhibit their ability to secrete inflammatory cytokines and form immune conjugates with T cells, thereby preventing further tissue and bone destruction.

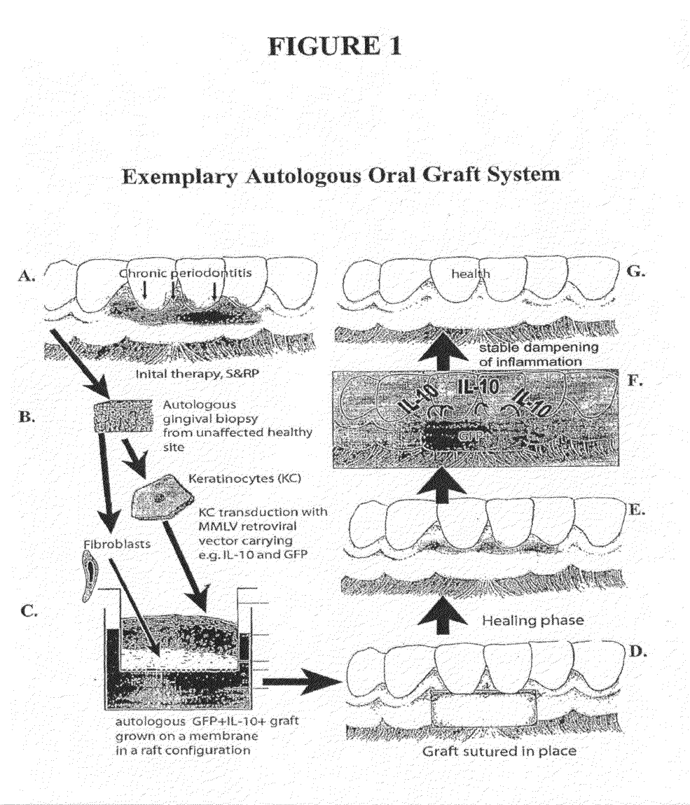

FIG. 1 provides an overview of one exemplary autologous oral graft system of the present invention, including one embodiment for preparing the autologous oral graft (FIGS. 1A-1C) and one embodiment for treating inflamed oral mucosa involved in chronic periodontitis with the autologous oral graft (FIGS. 1D-1G). FIG. 1A shows the oral cavity of a patient with inflamed oral mucosa tissue associated with chronic periodontitis. FIG. 1B shows a biological sample taken from an non-inflamed portion of the oral mucosa tissue and its separation into oral fibroblasts and oral keratinocytes which are subsequently transfected with a nucleic acid sequence encoding a therapeutic protein and a reporter gene. FIG. 1C shows a biological matrix with a layer of oral fibroblasts on top of the matrix and a layer of transfected oral keratinoctyes on top of the layer of fibroblasts to generate an exemplary autologous oral graft. FIG. 1D shows the autologous oral graft sutured in place on the oral mucosa of the patient. FIG. 1E shows the healing phase where at least part of the area of the oral mucosa that was inflamed is no longer inflamed. FIG. 1F shows therapeutic polypeptide expression (IL-10 is shown) and reporter protein expression (GFP is shown) by the transfected oral keratinocytes, where the therapeutic polypeptide promotes stable dampening of inflammation. Finally, FIG. 1G shows the patient's oral cavity after treatment.

### DETAILED DESCRIPTION OF THE INVENTION

Additional details on methods of making and using the autologous oral grafts of the present invention are provided below.

I. Generating Autologous Oral Grafts

The autologous oral grafts of the present invention maybe generated, for example, by the methods described below.

Autologous Oral Cell Procurement, Isolation, and Culturing

Autologous oral cells used to generate the oral grafts of the present invention are obtained from the patient to be treated. The patient will normally be identified as having one or more oral diseases that involve inflamed or damaged oral mucosa tissue or oral hard tissue. Cells from the patients oral cavity, such as oral mucosa cells, are then removed from a portion of the oral cavity that is not damaged or inflamed. Cells can be removed, for example, by numbing the tissue with local anesthesia, and removing a full-thickness oral mucosal biopsy (e.g., ˜5 mm pie-shaped wedge) of keratinized oral mucosa from a healthy oral mucosal site from the patient. Alternative methods may also be used, such as scraping a layer of cells off or using any type of swab. Preferably, a method is used that provides the desired cell types and a quantity that can be easily cultured. In certain embodiments, oral keratinoctyes are removed, while in other embodiments, oral fibroblasts are removed. The present invention is not limited by the type of cell that are removed and employed in the oral grafts.

In certain embodiments, the sample of cells is then treated with an antibiotic containing buffer and transferred aseptically into a vial (e.g. a liquid nitrogen vial). The vial may then be mailed (e.g., with dry ice) or otherwise delivered preferably to a graft manufacturing facility or other type of lab equipped to store or further process the cells. In other embodiments, the retrieved cells are initially placed in culture media (e.g., to expand the number of cells), and then delivered to a graft manufacturing facility or other type of lab.

In particular embodiments, the oral cells (e.g. oral mucosal tissue cells) are processed further (e.g. at the graft manufacturing facility). For example, if the cells are frozen, they are first thawed and then diced and/or enzymatically digested through standard procedures. Next, if oral keratinoctyes and oral fibroblasts are the desired cells types, the keratinocytes and fibroblasts are isolated using standard procedures (see, e.g., Izumi et al, Int J Oral Maxillofac Surg. 2003 April; 32(2):188-97, herein incorporated by reference in its entirety). Primary isolated keratinoctyes and fibroblasts are normally grown separately using standard tissue culture protocols.

In certain embodiments, the cells are frozen in liquid nitrogen and stored in a oral tissue bank, in the event that more grafts are needed, for example if the procedure needs to be repeated at other sites or the same site or if the initial graft fails to take.

In certain embodiments involving keratinocytes, the primary human oral keratinoctyes are grown in submerged cultures in the presence of a feeder layer of mitotically inactivated 3T3 cells (Rheinwald and Green, Cell 1975; 6: 331-344, herein incorporated by reference in its entirety), using keratinoctye medium described by Wu et al. (Cell 1982; 31: 693-703, herein incorporated by reference in its entirety) or similar media. In some embodiments, the keratinoctye media contains a mixture of about 3:1 Ham's F12 medium and Dulbecco's Modified Eagle Medium (DMEM), supplemented with 5% fetal calf serum, 5 μg/ml insulin (Sigma, St Louis, Mo.), 0.5 μg/ml hydrocortisone (Sigma), 0.1 nM cholera toxin (ICN Biochemicals, Cleveland, Ohio), 100 ng/ml epidermal growth factor (Chiron, Emeryville, Calif.), 10 μg/ml penicillin, and 10 μg/ml streptomycin (Sigma). In particular embodiments involving fibroblasts, human oral fibroblasts are maintained and cultured, for example, in Dulbecco's Modified eagle medium (DMEM) supplemented with 10% fetal bovine serum, 10 μg/ml penicillin, and 10 μg/ml streptomycin (Sigma), or similar media.

Transfected or Non-Transfected Oral Cells

In certain embodiments, at least one type of oral cell employed with the oral graft is transfected with a nucleic acid sequence encoding a therapeutic protein. In some embodiments, no transfection (or other modification) of oral cells is implemented and the cells are grown in a graft configuration as described and used to treat recession. In some embodiments, no expression vector is employed, and the naked nucleic acid is transfected by itself, while in preferred embodiments, the nucleic acid sequence is contained in an expression vector. As noted above, the present invention is not limited by the type of cell that is transfected. For example, any type of cell from the oral cavity of a patient may be employed as the cells to be transfected. Preferably, the transfected cells are oral fibroblasts or oral keratinocytes.

The present invention is not limited by the type of therapeutic polypeptide encoded by the nucleic acid. In certain embodiments, the therapeutic polypeptide comprises an anti-inflammatory cytokine (e.g., those cytokines that are able to inhibit the synthesis of IL-1, tumor necrosis factor (TNF), and other major proinflammatory cytokines or cytokine that can shift the T cell effector response towards Th1 (e.g. IFNγ, IL-12) or Th2 (e.g. IL-10, IL-4, IL-13)). In particular embodiments, the therapeutic polypeptide comprises an inhibitor of soft tissue proteolysis or inhibitor of matrix metalloproteinases (e.g., TIMP-1, TIMP-2, TIMP-3, or TIMP-4). In other embodiments, the therapeutic polypeptide comprises an inhibitor of bone resorption (OPN). In some embodiments, the therapeutic polypeptide comprises a stimulator of bone or other hard or soft tissue growth or regeneration (e.g. parathyroid hormone (PTH), bone morphogenetic protein (BMP), platelet-derived growth factor (PDGF-AB, PDGF-BB), transforming growth factor-beta1 (TGFβ-1), insulin-like growth factor-I (IGF-1), fibroblast growth factor-basic (FGF-b), epidermal growth factor (EGF), vascular endothelial growth factor (VEGF). The nucleic acid sequence and protein sequence of these proteins can be downloaded from services such as GENBANK, available through PUBMED. In further embodiments, the therapeutic polypeptide comprises an inhibitor of DC/T cell activation. In certain embodiments, the nucleic acid sequence encodes a cytokine. Examples of cytokine genes (with corresponding GENBANK accession numbers) include, but are not limited to, interleukin-1 (alpha, NM_{—}000575; beta, NM_{—}000576), interleukin-2 (NM_{—}000586), interleukin-4 (NM_{—}000589), interleukin-6 (NM_{—}000600), interleukin-7 (NM_{—}000880), interleukin-10 (NM_{—}000572), interleukin-12 (12A, NM_{—}000882; 12B, NM_{—}002187), interferon-alpha (NM_{—}024013), interferon beta (NM_{—}002176), interferon-delta, tumor necrosis factor alpha (NM_{—}000594) and beta (NM_{—}009588), granulocyte-macrophage colony stimulating factor (GM-CSF), and granulocyte colony stimulating factor (G-CSF). Other exemplary human therapeutic proteins (with nucleic acid encoding accession numbers) include, but are not limited to: TGF-beta (X02812), IL-IRA (M63099), vasoactive intestinal peptide (BC009794), CTLA-4 (BC074893), SHIP (X98429), IRAK-M (BC069388), SOCS 1 (U88326), SOCS 2 (AF159164), SOCS 3 (AB004904), SOCS 5 (AB014571), sTNFRI, STNFRII, sIL-1RII, mIL-1RII, and IL-18BP.

Determining whether a particular candidate nucleic acid sequence would be a suitable therapeutic protein for treating a particular oral disease with the oral grafts of the present invention can be accomplished, for example, by repeating Example 1 with a therapeutic sequence besides IL-10. If the candidate therapeutic protein is shown to reduce tissue inflammation or damage for a particular oral disease, then the candidate therapeutic protein is determined to be a therapeutic protein.

The present invention is also not limited by the type of expression vector used to transfect the oral cells that are used in the autologous oral graft. Any type of suitable vector may be employed, including, but not limited to, plasmids and viral vectors. Those skilled in the art will recognize and appreciate that other vectors are suitable for use with methods and compositions of the present invention. Indeed, the present invention is not intended to be limited to the use of the recited vectors, as such, alternative means for transfecting the oral cells of the present invention are contemplated, or no transfection of oral cells. For example, in various embodiments, the oral cells are transfected with retrovirus vectors and herpes virus vectors, plasmids, cosmids, artificial yeast chromosomes, mechanical, electrical, and chemical transfection methods, and the like. A number of viral vectors are known in the art and may be used for transfection including, but are not limited to, the following: adenoviral vectors; second generation adenoviral vectors; gutted adenoviral vectors; adeno-associated virus vectors; and lentiviral vectors. In certain embodiments, the oral cells are not transfected and/or modified at all.

In certain embodiments, a second nucleic acid sequence is transfected into the oral cells to serve as a reporter (e.g. to allow one to determine if the cells are successfully expressing the therapeutic protein). The present invention is not limited by the type of reporter gene employed. In certain embodiments, the reporter protein (expressed by the reporter gene) allows non-invasive monitoring of expression of the therapeutic polypeptide (e.g., expression of the therapeutic polypeptide can be monitored without removing the autologous oral graft from its position on the oral mucosal tissue of the patient). In particular embodiments, the reporter protein is selected from green fluorescent protein (GFP), firefly luciferase (FL), and beta-glucuronidase. In further embodiments, expression of the reporter protein is monitored, without removing the autologous oral graft from the oral mucosa tissue, with a light detection cooled charge coupled device (CCCD) or with a UV light, or with a fluorescence detector.

Biocompcatible Matrixes

As described above, one component of the autologous oral grafts of the present invention is a biocompatible matrix that can provide a support for cells, such as fibroblasts and keratinoctyes. The present invention is not limited by the type of biocompatible matrix that is employed. In certain embodiments, the biocompatible matrixes have at least one, and preferably all five of the following characteristics: 1) it is a polymer that is non-antigenic, non-animal-, non-human-tissue derived; 2) it permeable to cell nutrients, facilitating cell growth and adherence; 3) it tolerates in vitro cell culture and does not resorb or lose its handling characteristics for the length of time it takes to grow cells on the matrix (e.g., ˜14 days) and for up to 1 week after surgical placement; 4) the handling characteristics facilitate ease of surgical placement; and 5) it can be ringed with pre-fab holes for ease of suturing to the recipient bed. In certain preferred embodiments, the biocompatible matrix comprises a membrane. In other preferred embodiments, the biocompatible matrix is bioresorbable. In particular embodiments, the biocompatible matrix comprises polylactic acid and/or polyglycolic acid.

Generating the Autologous Oral Graft

The transfected oral cells, biocompatible matrix, and, in some embodiments, a second type of oral cell, are then combined to generate autologous oral grafts. The autologous oral grafts may be prepared using methods know in the art. For example, methods of culturing cells in biocompatible matrixes are provided in the following references: Ueda et al., Annals of Plastic Surgery, 27(6), 540-549, 1991; Izumi et al., Int. J. Oral. Maxillofax. Surg., 2003, 32:188-197; and Garlick and Fenjves, Crit. Rev. Oral. Biol. Med., 7(3):204-221, 1996, all of which are herein incorporated by reference in their entireties. One particular embodiment for generating an autologous oral graft using a tube type configuration is described in Example 1 below. Certain embodiments involves storing the cells after they have been grown in an “oral tissue bank,” so that they can be used again for that patient should the need arise

II. Treating Oral Cavity Diseases

The autologous oral grafts of the present invention may be used to treat oral cavity diseases, particularly those characterized by tissue inflammation or damage, or to treat oral caries characterized by damaged enamel, dentin, or cementum. In preferred embodiments, the oral cavity diseases are characterized by infiltration of the oral mucosa with activated, maturing dendritic cells and T cells.

Patients with chronic generalized or localized periodontitis are routinely treated by standard initial therapy, including scaling and root planning and oral hygiene instruction, as performed by the dentist or hygienist in a dental or periodontal practice. A certain number of patients do not respond adequately after 4-6 weeks, as evidenced by continued inflammation, bleeding on probing, and loss of clinical attachment. In certain embodiments, such patients, rather than being treated by more invasive procedures such as oral mucosal flap/osseous or regenerative surgical procedures, may be treated with the autologous oral grafts of the present invention. In other embodiments, such as in the case of moderate to severe periodontitis, the autologous oral grafts of the present invention may be administered after completion of the healing phase of oral mucosal flap/osseous or regenerative surgical procedures (i.e. the standard of care for moderate to severe periodontitis) in order to dampen inflammation in the long term. In other embodiments, the autologous oral grafts of the present invention are employed as first line therapy in order to treat oral diseases or are used to treat gingival recession.

As shown in FIG. 1D, the autologous oral graft may be sutured to the gingiva tissue in the oral cavity of the patient. Any other type of attachment means and locations, however, may be employed. For example, the autologous oral grafts may be attached by glue, pins, screws, or held in place by a mouthpiece until such graft heals. Also for example, the autologous oral graft may be attached to various tissues or region of the oral cavity, including, but not limited to, the palate tissue, the gingiva tissue, the buccal mucosa tissue, the tongue, the floor of the mouth tissue, or one or more teeth.

### EXAMPLES

The following examples are provided in order to demonstrate and further illustrate certain preferred embodiments and aspects of the present invention and are not to be construed as limiting the scope thereof.

### Example 1

### Generating Autologous Oral Grafts for Treating Oral Disease

This Example describes the generation of an autologous oral graft for treating oral diseases, such as those involving tissue inflammation or damage. A subject with an oral disease such as chronic periodontitis, is identified as needing treatment. Under local anesthesia, a dental surgeon removes an autologous full-thickness oral mucosal biopsy (e.g., ˜5 mm pie-shaped wedge) of keratinized oral mucosa from a healthy oral mucosal site from the patient. A single resorbable suture is placed at the biopsy site. The oral mucosal biopsy is rinsed with antibiotic containing buffer, and immediately transferred aseptically into liquid nitrogen vial provided to the dentist by the clinical lab, and mailed overnight to a graft manufacturing facility.

Once at the graft manufacturing facility, the autologous oral mucosal tissue is thawed, diced and/or enzymatically digested through standard procedures, and keratinocytes (KC) and fibroblasts (FB) are isolated, as reported in the literature (Izumi et al, Int J Oral Maxillofac Surg. 2003 April; 32(2):188-97, herein incorporated by reference in its entirety). Primary isolated KC and FB are grown separately using standard tissue culture protocols. Samples of KC and FB are stored in liquid nitrogen (oral tissue bank) for subsequent use f the need arises.

Primary human oral KC are grown in submerged cultures in the presence of a feeder layer of mitotically inactivated 3T3 cells (Rheinwald and Green, Cell 1975; 6: 331-344, herein incorporated by reference in its entirety), using KC medium described by Wu et al. (Cell 1982; 31: 693-703, herein incorporated by reference in its entirety). The KC medium contains a mixture of 3:1 Ham's F12 medium and Dulbecco's Modified Eagle Medium (DMEM), supplemented with 5% fetal calf serum, 5 μg/ml insulin (Sigma, St Louis, Mo.), 0.5 μg/ml hydrocortisone (Sigma), 0.1 nM cholera toxin (ICN Biochemicals, Cleveland, Ohio), 100 ng/ml epidermal growth factor (Chiron, Emeryville, Calif.), 10 μg/ml penicillin, and 10 μg/ml streptomycin (Sigma). Human oral FB are maintained and cultured in Dulbecco's Modified eagle medium (DMEM) supplemented with 10% fetal bovine serum, 10 μg/ml penicillin, and 10 μg/ml streptomycin (Sigma).

The KCs, genetically engineered to secrete human IL-10 and green fluorescent protein (GFP), are prepared using recombinant retrovirus transduction. The various constructs described below may be used to transfect 293 GP retroviral packaging cells using the calcium phosphate co-precipitation protocol (Burns et al., Proc Natl Acad Sci USA 1993; 90: 8033-8037; and Pear et al., Proc Natl Acad Sci USA 1993; 90: 8392-8396, both of which are herein incorporated by reference), but other methods to induce transfer of genes into KC without invoking viral transfection, including chemical, physical methods could also be used (see, e.g., Garlick and Fenjives, Crit Rev Oral Biol Med. 1996; 7(3):204-21, herein incorporated by reference). The engineered KCs in this Example secrete IL-10 (e.g., for dampening inflammation), but other biological mediators and negative regulators of inflammation and of intracellular signaling could also be employed, including, but not limited to: TGF-β, IL-1RA, vasoactive intestinal peptide (VIP), CTLA-4, SHIP, SOCS-1, IRAK-M, and other negative regulators, the sequences of which are obtained from NCBI website (“www.” followed by “nobi.nlm.nih.gov/”).

After 2 days the KCs isolated from the patient are passaged into a medium containing puromycin (1 mg/ml) and are maintained in this selective medium. Resistant producer cells are transiently transfected with pHCMVG DNA encoding vesicular somatitis virus-glycoprotein G (see, e.g., Burns et al., Proc Natl Acad Sci USA. 1993; 90:8033-8037, herein incorporated by reference). Forty-Eight hours after transfection the producer cells lyse and release the replication incompetent retroviruses into the supernatant. The supernatants are filtered through 0.45-mm poresize filter (Gelman science, Ann Arbor, Mich., USA) and are either stored as is at −70° C. or concentrated further by ultra-centrifuging using a protocol described in Burns et al. Retroviral transfection is achieved by adding known volumes of viral supernatant to a predetermined number of oral mucosal KCs in six well plates in the presence of 8 mg/ml poly-brene (Sigma, St Louis, Mo., USA) at 37° C. The retroviral construct (IL-10-EGFP-MMLV-vector) consists of bicistronic replication incompetent MMLV retrovial vector carrying the human or viral IL-10 gene and the EGFP (or other suitable reporter gene) interrupted by an internal ribosome entry site (IRIS). Human or v IL-10 is translated off the viral LTR and EGFP is read of the IRES in this transcript (see, e.g., Lu Z, Ghazizadeh D, Host immune responses in ex vivo approaches to cutaneous gene therapy targeted to keratinocytes. Exp Dermatol. 2005 October; 14(10):727-35, herein incorporated by reference). After 2-3 hours, fresh medium is added and 2 days after transfection, cells are either passed or harvested for FACS analysis. The percentage of cells positive for GFP are then used to calculate viral titers, which should range from about 1.5×10⁷-8×10⁷ infective units.

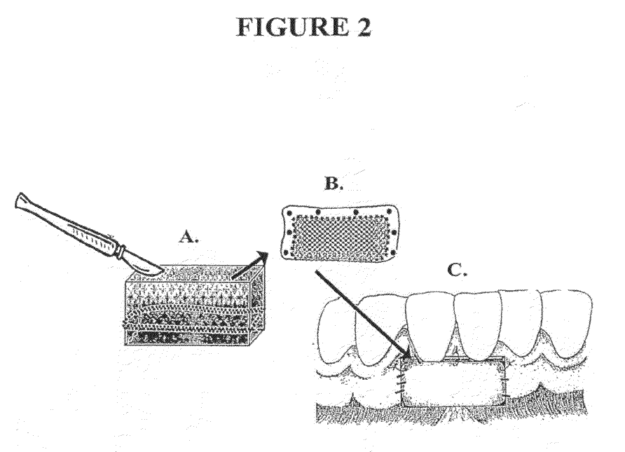

The autologous oral graft is then prepared using, for convenience, a tube configuration. In particular, a rectangular tube, that is open at both ends, and which is composed of a biocompatible polymer (polypropylene in this Example), is employed along with a bioresorbable membrane. The bioresorbable membrane in this Example is composed of polylactic acid. This membrane has a rectangular shape and is about 25 mm in length, about 15 mm in width, and is about 0.1 mm (100 microns) thick. The membrane has pores that are about 8 μm in size and also contains a number of pre-formed suture holes (e.g. as shown in FIG. 2D). The membrane is attached at or near one end of the rectangular tube and sealed around the edge with cyanoacrylate cement or other suitable adhesive, thereby forming a cup configuration with the membrane as the bottom of the cup and the rectangular tube forming the side walls of the cup.

Cells are then grown on the bioresorbable membrane inside of the cup using a modification of a published protocol (Nolte et al., Arch Dermatol Res. 1993; 285(8):466-74, herein incorporated by reference in its entirety). Briefly a matrix of FB (2×10⁶ cells/ml) containing type I collagen (2 mg/ml, DME, 1M NaOH (2 μl/ml) and fetal bovine serum (10%) is layered on the bioresorbable matrix. The cup is then submerged in KC medium, as described in Wu et al. (Cell 1982; 31: 693-703, herein incorporated by reference in its entirety) and fed for a week. Cultured genetically engineered or genetically unaltered KC (10⁷ cells/ml) (prepared as described above) are layered on top of the FB matrix and grown in the cup, which is placed on 1 mm spacers that hold it in place, but slightly elevated from the bottom of a 24 well tissue culture plate. Enough keratinocyte medium is added to the tissue culture well to cover the bottom of the cup and some is added to the inside of the cup to cover the cells and a tissue culture plate cover is placed. After 4 days cultures are raised to air-liquid interface and fed only from the bottom of the well with the KC medium (raft configuration).

### Example 2

### Treating Oral Disease with an Autologous Oral Graft

This Example describes the treatment of an oral disease with an autologous oral graft. In particular, a patient with chronic periodontitis is treated with the autologous oral graft prepared as described in Example 1. Initially, a surgeon prepares the graft recipient bed using a standard procedure for a free-oral mucosal graft. The autologous oral graft from Example 1 is removed from the vial by rimming it with a sterile 15 c scalpel while holding the oral graft with cotton forceps to prevent the graft from curling up prior to insertion. Next, the oral graft is inserted into the patient's mouth at the appropriate site along the gum tissue and is sutured in place with 5-0 gut chromic gut suture using a Castroviejo needle holder. Alternatively, the comers of the autologous oral graft are tacked down with surgical adhesive to stabilize the graft, and then it is sutured down tightly to the recipient bed. The autologous oral graft is stably bound down to the periosteum such that there is not movement when pulling the lips or cheeks. A surgical dressing of choice is placed to protect the oral graft during the initial healing phase (approx 10-14 days).

The success of the grafting procedure and the stability of the gene transduction with time are monitored by use of a UV light for green fluorescent protein (GFP). Saliva and crevicular fluid are collected and sent to the graft manufacturing facility, or other lab, for analysis of GFP and the gene product (e.g. IL-10). At any time, graft can be removed surgically and complete removal monitored by UV fluorescence. It is noted that the Patients' cells may be archived in liquid nitrogen by the manufacturing facility, or other lab, in the event re-treatment is necessary.

### Example 3

### Autologous Oral Grafts with Untransfected Oral Keratinocytes

In order to generate an autologous oral graft that employs untransfected oral keratinocytes, Example 1 could be repeated, while omitting the transfection steps. An oral graft generated in this manner could be employed to treat a patient with gingival recession, the result of damage to the oral mucosa by processes as described in Example 2. Treatment of oral disease with such un-transfected oral keratinocytes serves to help reduce the damage to the oral mucosa tissue or the oral calcified tissue in the oral cavity of the patient (e.g., by providing keratinized tissue and by secreting endogenous factors, such as cytokines and other proteins, that serve to reduce damage to the oral cavity).

All publications and patents mentioned in the above specification are herein incorporated by reference. Various modifications and variations of the described oral graft devices, compositions, methods, systems, and kits of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in art are intended to be within the scope of the following claims.

I claim:

1. A method of treating oral cavity disease comprising:
a) inserting an autologous oral graft into the oral cavity of a patient, wherein said oral cavity of said patient comprises oral mucosa tissue and oral hard calcified tissue, wherein a portion of said oral mucosa tissue is inflamed or damaged oral mucosa tissue as a result of an oral cavity disease or wherein a portion of said oral calcified tissue is damaged, and wherein said autologous oral graft comprises: i) a biocompatible matrix, and ii) cultured oral keratinocytes generated from oral keratinocytes taken from said patient, wherein said cultured oral keratinocytes comprise an expression vector comprising a first nucleic acid sequence encoding a therapeutic polypeptide; and
b) attaching said autologous oral graft to said oral mucosa tissue, or said oral hard calcified tissue, in said oral cavity of said patient such that said therapeutic polypeptide is expressed and secreted from said cultured oral keratinocytes.

2. The method of claim 1, wherein said expression vector further comprise a second nucleic acid sequence encoding a reporter protein, wherein said reporter protein allows monitoring of expression of said therapeutic polypeptide.

3. The method of claim 2, further comprising step c) detecting expression of said reporter protein without removing said autologous oral graft from said oral mucosa tissue thereby determining if said therapeutic polypeptide is expressed.

4. The method of claim 1, wherein said autologous oral graft further comprises cultured fibroblasts, wherein said cultured fibroblasts are generated from original fibroblasts taken from said patient.

5. The method of claim 1, wherein said inflamed or damaged oral mucosa tissue comprises gingiva tissue.

6. The method of claim 1, wherein said biocompatible matrix is bioresorbable.

7. The method of claim 1, wherein said biocompatible matrix comprises pores.

8. The method of claim 1, wherein said biocompatible matrix has a length between about 15 mm and 35 mm.

9. The method of claim 1, wherein said biocompatible matrix has a width between about 5 mm and 15 mm.

10. The method of claim 1, wherein said biocompatible matrix has a thickness between about 0.05 mm and 0.15 mm.

11. The method of claim 1, wherein said biocompatible matrix comprises a plurality of pre-formed suture holes.

12. A method of making an autologous oral graft comprising;
a) culturing oral keratinocytes taken from the oral cavity of a patient to generate cultured oral keratinocytes, wherein said oral cavity of said patient comprises oral mucosa tissue and oral hard calcified tissue, wherein a portion of said oral mucosa tissue is inflamed or damaged oral mucosa tissue as a result of an oral cavity disease, or wherein a portion of said oral hard calcified tissue is damaged, and wherein said oral keratinocytes are taken from a portion of said oral mucosa tissue that is not inflamed or damaged;
b) transfecting said cultured oral keratinocytes with an expression vector comprising a first nucleic acid sequence encoding a therapeutic polypeptide; and
c) combining said cultured oral keratinocytes with a biocompatible matrix to generate an autologous oral graft, wherein said autologous oral graft is configured to be attached to said oral mucosa tissue, or said oral hard calcified tissue, of said patient such that said therapeutic polypeptide is expressed and secreted from said cultured oral keratinocytes.

13. The method of claim 12, wherein said expression vector further comprises a second nucleic acid sequence encoding a reporter protein, wherein said reporter protein allows monitoring of expression of said therapeutic polypeptide.

14. The method of claim 12, further comprising the step of culturing original oral fibroblasts taken from the oral cavity of said patient to generate cultured oral fibroblasts.

15. The method of claim 14, wherein said cultured oral fibroblasts are combined with said cultured oral keratinocytes and said biocompatible matrix to generate said autologous oral graft.

16. A method of treating oral cavity disease comprising:
a) inserting an autologous oral graft into the oral cavity of a patient, wherein said oral cavity of said patient comprises oral mucosa tissue and oral hard calcified tissue, wherein a portion of said oral mucosa tissue is inflamed or damaged oral mucosa tissue as a result of an oral cavity disease or wherein a portion of said oral calcified tissue is damaged, and wherein said autologous oral graft comprises: i) a biocompatible matrix, and ii) cultured oral keratinocytes generated from oral keratinocytes taken from said patient; and
b) attaching said autologous oral graft to said oral mucosa tissue, or said oral hard calcified tissue, in said oral cavity of said patient such that the damage to said oral mucosa tissue or said oral calcified tissue in said oral cavity of said patient is halted or reduced.

17. The method of claim 16, wherein said autologous oral graft further comprises cultured fibroblasts, wherein said cultured fibroblasts are generated from original fibroblasts taken from said patient.

18. A method of making an autologous oral graft comprising;
a) culturing oral keratinocytes taken from the oral cavity of a patient to generate cultured oral keratinocytes, wherein said oral cavity of said patient comprises oral mucosa tissue and oral hard calcified tissue, wherein a portion of said oral mucosa tissue is inflamed or damaged oral mucosa tissue as a result of an oral cavity disease, or wherein a portion of said oral hard calcified tissue is damaged, and wherein said oral keratinocytes are taken from a portion of said oral mucosa tissue that is not inflamed or damaged, and
b) combining said cultured oral keratinocytes with a biocompatible matrix to generate an autologous oral graft, wherein said autologous oral graft is configured to be attached to said oral mucosa tissue, or said oral hard calcified tissue, of said patient such that the damage to said oral mucosa tissue or said oral calcified tissue in said oral cavity of said patient is halted or reduced.

19. The method of claim 18, further comprising the step of culturing original oral fibroblasts taken from the oral cavity of said patient to generate cultured oral fibroblasts.

20. The method of claim 19, wherein said cultured oral fibroblasts are combined with said cultured oral keratinocytes and said biocompatible matrix to generate said autologous oral graft.
